(19) United States
(12) Patent Application Publication
(10) Pub. No.: US 2013/0079826 A1
(43) Pub. Date: Mar. 28, 2013

(54) SPINAL ROD AND BONE SCREW CAPS FOR SPINAL SYSTEMS ASSEMBLIES

(71) Applicant: Peter M. Simonson, Longboat Key, FL (US)

(72) Inventor: Peter M. Simonson, Longboat Key, FL (US)

(21) Appl. No.: 13/243,602

(22) Filed: Sep. 23, 2011

(51) Int. Cl. A61B 17/70; B23P 11/00

(52) U.S. Cl. 606/264; 29/428

(57) ABSTRACT

A threaded cap or c-clip that attaches to a spinal rod or bone screw. Such a threaded cap or c-clip prevents slippage of the spinal system assemblies along or off their rods or bone screws during intra- or post-operative periods.

### FIELD OF INVENTION

The present invention relates to devices, implants and tools used in orthopedic spinal surgical procedures. Specifically, the invention improves spinal system assemblies with a threaded cap or c-clip for securing spinal rods and bone screws.

### BACKGROUND OF THE INVENTION

Back pain is a commonly reported medical aliment. It is most frequently associated with degenerative changes in the spinal vertebra. Most of the 30 million people in the U.S. reporting back pain each year resolve their pain with conservative treatment, or simply rest and exercise. Nonetheless, approximately 15 percent, or 4.5 million, fail conservative therapy and are left with debilitating pain. Out of these, approximately 500,000 people opt for spinal surgery. In addition to alleviating pain, spinal surgery seeks to minimize damage to adjacent supportive muscle and skeletal components.

Several techniques and systems have been developed for correcting and stabilizing the spine and, where appropriate, facilitating spinal fusion. Over the years, spinal and orthopedic implants have evolved toward progressively stronger, stiffer and better devices, as it is presumed that increased construct rigidity optimizes bone fusion and provides more rapid and robust healing. The most widely used systems use a bendable rod that is placed longitudinally along the length of the spine. Such a rod is bent to follow the normal curvature of the spine whether it is the normal kyphotic curvature for the thoracic region or the lordotic curvature for the lumbar region. In such a procedure, a rod is attached through intermediate connectors to various vertebrae along the length of the spinal column by a number of bone screws threaded into the pedicles of the vertebral bodies. When stabilized, the vertebra is decortified during the fusion process where the outer cortical bone is removed to provide a foundation for bone grafts. Over time, these bone grafts fuse the damaged vertebrae together.

A good example of a rod spinal fixation system is the TSRH® Spinal System sold by Medtronic Sofamor Danek Inc. When introduced, the TSRH® Spinal System was a significant advance over prior systems. FIG. 1 shows a spinal system assembly 2, more specifically, a current design of the variable height TSRH® Spinal System described in U.S. Pat. No. 5,643,263 to Simonson. The spinal system assembly 2 comprises a rod 4, a bone screw 6 and a connector assembly 8, including a rod washer 10 with an oversized rod washer aperture 12 and bone screw washer 14 also with an oversized bone screw washer aperture 16. Like the earlier TSRH®Spinal System, the rod washer 10 has radially splined ridges 18 (FIG. 1A) facing the bone screw washer 14. The Simonson design however added another complementary radially splined ridge surface 19 to the bone screw washer 14 (FIG. 1C). In combination, the connector assembly 8 moves at variable angles relative to the spinal rod with both the rod washer 10 and bone screw washer 14 able to rotate 180° with respect to one another. The connector assembly 8 between the rod 4 and bone screw 6 swivels up and down 24 over the bone screw shank 20 and horizontally 26 over the rod 4. With this type of connector, the rod 4 and bone screw 6 can be in differing angular positions. The one sized bone screw washer aperture 16 can also receive a bone screw shank 20 of various sizes and thicknesses using different sized interface washers thereby meeting any required distance between the rod 4 and bone screw 6. Adapting this type of connector assembly 8 to the TSRH® Spinal System allowed the bore diameters of both the rod 4 and bone screw 6 to be effectively reduced as the rod 4, rod washer 10, bone screw washer 14 and bone screw 6 are pressed together by a top-tightening set screw 28. When the rod and bone washers 10, 14 are properly positioned over the rod 4 and bone screw 6, the set screw 28 is tighten. When the set screw 28 forces the rod 4 toward the rod washer 10, the entire assembly becomes locked against any movement. Adjustments can then be made by loosening the set screw 28 and re-tightening it until the preferred position is reached. When properly adjusted, the set screw 28 is tightened and snapped off.

FIG. 1 also illustrates the rotation ability of the connector assembly 8. When a rod 4 is positioned in the aperture 12 of the rod washer and the shank 20 of the bone screw 6 is then positioned through the bone screw aperture 16 of the bone screw washer 14, the rod 4 and bone screw 6 rotate 30 (rod rotation), 32 (bone screw rotation) relative to each other since the rod and bone screw apertures 12, 16 of the rod washer 10 and the bone screw washer 14 are larger than the rod 4 and bone screw 6, respectively. With such flexibility between the rod 4 and a plurality of bone screws 6, the connector assembly 8 can be moved 26 along the rod 4 allowing the rod 4 and bone screw 6 to be in differing angular positions because the rod washer 10 and bone screw washer 14 can swivel 34 relative to each other. The linear distance between the rod and bone screw is also adjustable because of the variability provided by the apertures. With the ability of the connector assembly 8 to rotate with respect to the rod and bone screw washers, the washers allow the TSRH® Spinal System to also rotate in the sagittal plane 36.

Along with variable-sized rod and bone screw washers 10, 14, the TSRH® Spinal System can also undergo a 160° medial-lateral 38 adjustment. With this medial-lateral 38 ability, the TSRH® Spinal System can engage any laterally placed bone screws 6. This characteristic is particularly important for multi-level rod constructs between the vertebrae. With its radially splined ridges 18, various sized rod and bone screw washers 10, 14 and the smooth bone screw shank 20, the anatomic placement of pedicle screws can now be made with minimal rod contouring, thereby reducing any forced preloading or stressing of the screw-to-rod interface.

With the TSRH® Spinal System and its variable height capability, a single level rod construct can be easily placed throughout the spine. In otherwords, a bendable rod can easily be placed longitudinally along the entire length of the spine and bent to follow the normal curvature of the spine whether it is the normal kyphotic curvature for the thoracic region or the lordotic curvature for the lumbar region or both.

Once the degree of offset between the bone screws has been determined with the TSRH® Spinal System and the appropriate sized connectors, set screws are placed on the rod. Finger tightening the set screw holds the connector in position on the rod while allowing the splines to swivel and line up with the screw post. To facilitate the loading of the rod/connector on the screws, malleable nitinol screw extenders help guide the connectors to their proper alignment and allow engagement with the screws. The contoured rod is then sequentially advanced through each connector. After the construct has been assembled, segmental spinal distraction and compression is carried out. During such, the connector assembly glides up and down the rod where it can be provisionally tightened and retightened as final adjustment are carried out with a screw adjustment driver. While such adjustments are being made, the construct may come apart with the connector assembly coming off the bone screw. In most circumstances, these disassemblies occur during intra-operative adjustments and before the set screw is finally tighten. After reassembly and perhaps further minor adjustments, the surgeon performs final tightening.

FIG. 2 shows a TSRH® Spinal System assembly 2 with its rod 4 and connector assembly 8 in various configurations (solid, dotted and dashed lines). Intra-operatively, the optimal rod position 40 (solid line) is when the rod washer 10 is at an appropriate distance from end of the rod. Such an optimal rod position 40 allows a safe distance before the connector assembly 8 slips completely off the end of the rod 4. It is at this optimal rod position 40 where provisional tightening is performed. A well-placed and provisionally tightened Spinal System assembly 2 allows for minor slippage especially during intra-operative adjustments. On the other hand, movement further down the rod provides a more downward rod position 42 (dashed line) that is less than optimal. Instead, it may indicate slippage at the other end of the rod. The most disadvantageous rod position 44 (dotted line) is when the end of the spinal rod 4 is underneath 46 the set screw 28 of the rod washer. With this scenario, the possibility of the connector assembly 8 coming completely apart during surgery is high. Although not critical, such a scenario is inconvenient for the surgeon because he/she may have to re-assemble the connector assemblies. A need therefore exists to prevent such rod and connector disassemblies during the intra-operative period.

The same principal applies to the bone screw. FIG. 2 also shows a TSRH® Spinal System assembly 2 with its bone screw 6 and connector assembly 8 in various configurations (solid dotted and dashed lines). Intra-operatively, the optimal bone screw position 48 (solid line) is when the bone screw washer 14 is at an appropriate distance from end of the bone screw 6. Such an optimal bone screw position 48 allows a safe distance before the connector assembly 8 slips completely off the end of the bone screw 6. It is at this optimal bone screw position 48 where provisional tightening is performed. A well-placed and provisionally tightened spinal system assembly 2 allows for minor slippage especially during intra-operative adjustments. On the other hand, movement of the bone screw 6 further up provides a more upward bone screw position 50 (dashed line) that is less than optimal but not critical. The most disadvantegous position 52 (dotted line) is when the end of the bone screw 6 is underneath the bone screw washer 14. With this scenario, the possibility of the connector assembly 8 and especially the bone screw washer 14 coming completely apart is high. Although not critical, such a scenario is inconvenient for the surgeon because he/she may have to re-assembly the connector assemblies. A need therefore exists to prevent the connector assembly from coming apart during the intra-operative period.

As described, the suboptimal placement of the rod washer 10 or bone screw washer 14 along their rods 4 or bone screws 6 is inconvenient. If positioned poorly, such instrumentation may come apart during the operation but can be easily re-assembled and put back into place. Once fixed and in place, the set screw 28 is tightened and, after surgery, the spinal rod fixation implants become in most cases, permanent.

After surgery and within a patient's normal range of motion, there are of course micro-motions of the skeletal system. These micro-motions are constant and present throughout the spinal systems 2. In referring back to FIG. 2, such micro-motions start in the bone screw 6, proceed along the connector assembly 8 and travel to the rod 4. Over time, these micro-motions may lead to a progressive degradation of the spinal system assemblies 2 from their initially implanted state. During such degradation, there may be pressure or forces weakening the spinal system compression points. Over time, these pressures or forces may lead to movement or slippage along the rods 4 or, in worst-case scenario, metal fatigue and eventual breakage. A need therefore exists to mitigate any disassembly of spinal systems.

In summary, there is always a need in the industry for improvements in devices, implants and tools used in orthopedic spinal surgical procedures. The present invention helps to prevent the disassembly of spinal systems in both intra- and post-operative circumstances. There are also new tool embodiments to help place and remove such devices on spinal system assemblies.

### BRIEF SUMMARY OF THE INVENTIONS

The present invention provides improvements to bone screws, rods and tools used in orthopedic spinal surgical procedures. Specifically, the present invention improves the spinal system assemblies with a threaded cap, a c-clip and tools for securing and connecting such caps and c-clips to bone screws and spinal rods.

In one embodiment, the present invention is a cap or c-clip on either a bone screw or rod or both to prevent slippage and disassembly of spinal system assemblies along or off their respective rods or bone screws during intra-operative construction or post-operative stress. The cap or c-clip makes it more convenient for the surgeon to put together the spinal system assembly and thereby reduces his/her time during the operation. With such caps, spinal system assemblies will not come apart before spinal compression and final tightening of the set screw.

To attach a cap to the rod or bone screw, threads on the ends of spinal rods and/or bone screws may be used. With corresponding threads, a cap can be easily placed onto a rod or bone screw. Furthermore, a threaded cap can be removed to either disassemble or modify parts of spinal system assemblies.

Another preferred embodiment is similar to the first but is more easily placed and removed. This embodiment is a c-clip that snaps into a groove positioned at or near the end of spinal rods or bone screws. Such a groove is cut into and around the end of a bone screw or rod. In some circumstances, a c-clip may be preferable to the threaded cap because the rods and bone screws do not need to be threaded. There are two types of c-clip embodiments. One is a c-clip that is intended to be an alternative to the threaded cap. It contains a lip with a hole so that it can be easily pushed onto the spinal rod or bone screw thereby making it more easily and quickly placed. The other embodiment is a lighter c-clip similar to a spring and made of alloy metals. It may be used intra-operatively and removed when the set screw is tighten.

Like the earlier cap embodiment, the c-clip embodiments also allows the pre-operative construction of the spinal system assembly. When pre-assembled, the c-clip makes it more convenient for the surgeon and will reduce his/her time putting the assemblies together during the operation. With such c-clips, spinal system assemblies will not come apart before final spinal compression and tightening. Like the earlier cap embodiment, a c-clip can also prevent slippage and disassembly of the spinal system assemblies along or off their respective rods or bone screws either during intra-operative construction or post-operative stress.

In the long run, the present cap and c-clip embodiments may also serve as backup or fail-safe devices. Over time, loads or forces on the spinal implants weaken bone screws and their connectors along spinal rods. In rare instances, a bone screw may pull out from the vertebral body and start to pull the spinal system assembly apart. If the cap and c-clip embodiments are present, they may prevent or mitigate a total disassembly of the spinal system assembly by acting as end plates or stoppers to ensure the connectors or washers on either the bone screw or rod do not become detached from each other. Even if loosened, the connector assembly can still retain some of its support with either the cap or c-clip holding the connector assembly in place until such time when implants can be retighten, replaced or fusion is complete.

Over time, biological tissue, such as bone, also settles and grows into the apertures of spinal system assemblies. Such tissue migration can affect spinal system assembly performance and strength. As a result, there may be instances where it is advantageous, or perhaps necessary, to clean, remove or replace such assemblies or assembly parts. In contrast to permanent caps, removable caps and/or c-clips allows the surgeon to do so. On the other hand, permanent caps hamper the disassembly of spinal systems and make it much more difficult for removal of spinal systems.

In rare instances, when no torque is applied or when the torque is below specifications, especially during poor surgical procedures, the caps and c-clips can also mitigate any further damage or disassembly of the spinal system assembly until another surgical procedure fixes it.

Working in combination with the present cap or c-clip inventions, additional embodiments include tools to facilitate the placement or removal of such caps and c-clips from spinal rods and bone screws.

### BRIEF DESCRIPTION OF THE DRAWINGS

FIG. 1 shows a perspective view of the TSRH® Spinal System described in U.S. Pat. No. 5,643,263 to Simonson with FIGS. 1A, 1B and 1C showing more detail of the rod and bone screw washers.

FIG. 2 shows a spinal system assembly with its connector assembly at various positions along the rod and bone screws.

FIG. 3A shows a perspective view of a spinal system assembly with a threaded rod and cap.

FIG. 3B is an expanded view of the preferred threaded cap embodiment shown in FIG. 3A.

FIG. 3C is another embodiment of the threaded cap with flat sides for threading and tightening by a wrench.

FIG. 4 shows various attachment and driver tool embodiments for use with the threaded cap. FIG. 4A shows a hex cap and driver section. FIG. 4B shows a pin hole cap embodiment. FIG. 4C shows the pin hole cap embodiment with its corresponding driver.

FIG. 5A shows a close-up of a c-clip that can be attached to either a spinal rod or bone screw.

FIG. 5B is similar to FIG. 5A but shows the c-clip with an attachment lip.

FIG. 5C shows a c-clip attachment tool.

FIG. 6 shows the preferred threaded cap (FIG. 6A) and c-clip (FIG. 6B) embodiments on a bone screw.

FIG. 7 shows another c-clip embodiment. FIG. 7A is a top view of the light c-clip.

FIG. 7B is a side view of the light c-clip. FIG. 7C shows an expander tool to use in snapping the light c-clip onto either a rod or bone screw.

FIG. 8 illustrates the preferred cap and c-clip embodiments at various positions on the spinal system assembly.

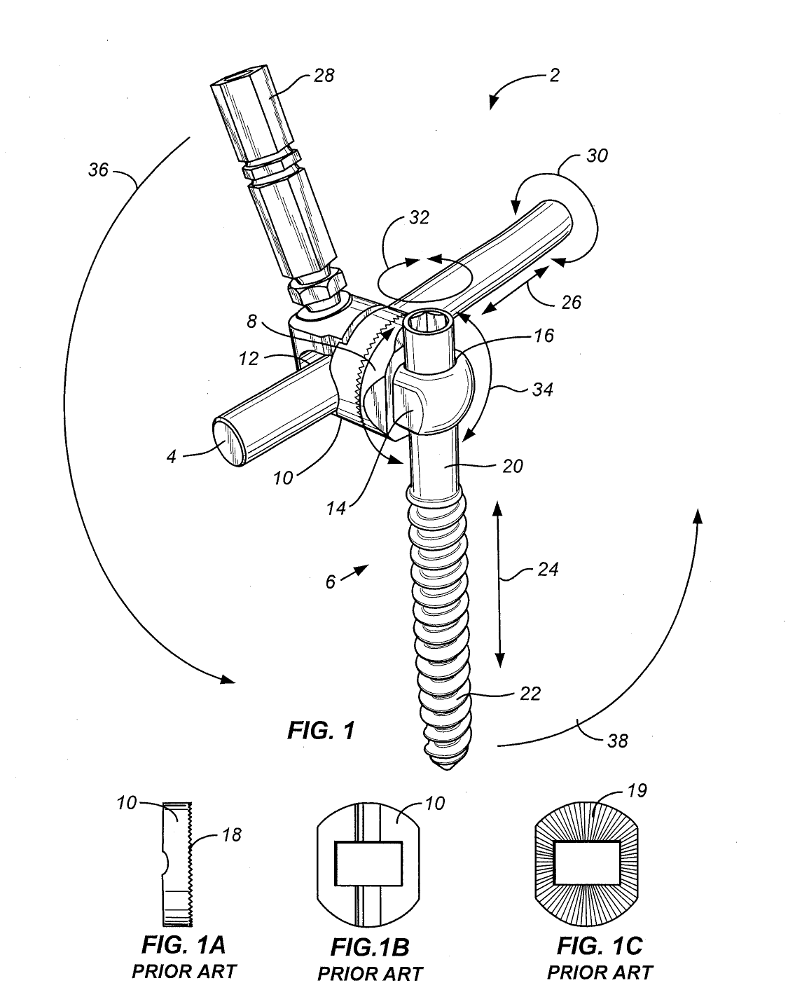

### DETAILED DESCRIPTION OF THE INVENTION

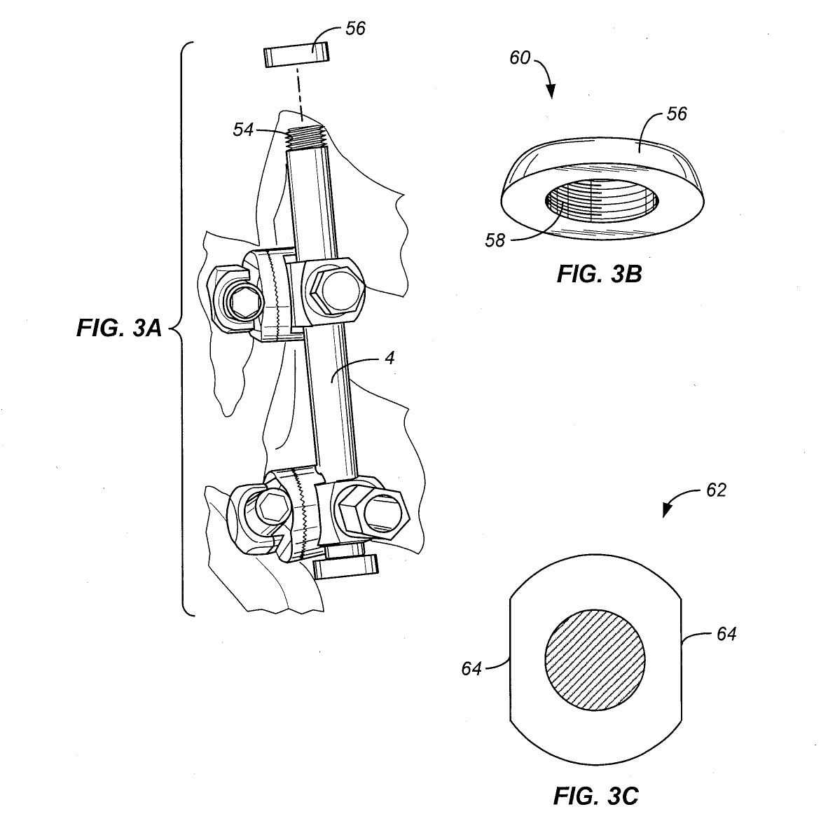

Referring to FIG. 3A, a threaded cap 56 is shown which screws on to spinal rod threads 54 on the end of spinal rod 4. As shown in FIG. 3B, the cap 56 has a threaded aperture 58 into which spinal rod threads 54 from a spinal rod 4 are screwed. Typically, the spinal rod 4 has male threads, while the cap 56 has female threads. However, the gender of the spinal rod 4 and cap 56 may be reversed where the spinal rod 4 comprises female threads and the cap 56 has male threads. One preferred embodiment is a smooth and round cap 60. Such a smooth and round cap 60 can be easily placed onto the spinal rod 4, screwed into a spinal rod threads 54 and finger-tightened by a surgeon. If a more secure cap is desired, a wrench cap 62 with flat sides 64 such as shown in FIG. 3C may be used. A wrench cap 62 is similar to a washer or nut with flat sides. In this embodiment, the two flat sides 64 allow a small hand wrench to torque the wrench cap 62 a little tighter than finger tightening. Although a four-sided wrench cap is possible, a two-sided embodiment is preferred so as not to have the sharp edges of a four-side washer or nut.

The cap 56 is preferably made of the same metal as its respective rod 4. In most cases, the preferred metal is stainless steel or titanium. It is preferred that they are made of the same metal as to avoid metal galvanization. When rods and bone screws are made from composite plastic, such as PEEK (polyether ether ketone), then the cap 56 can also be made of such plastics.

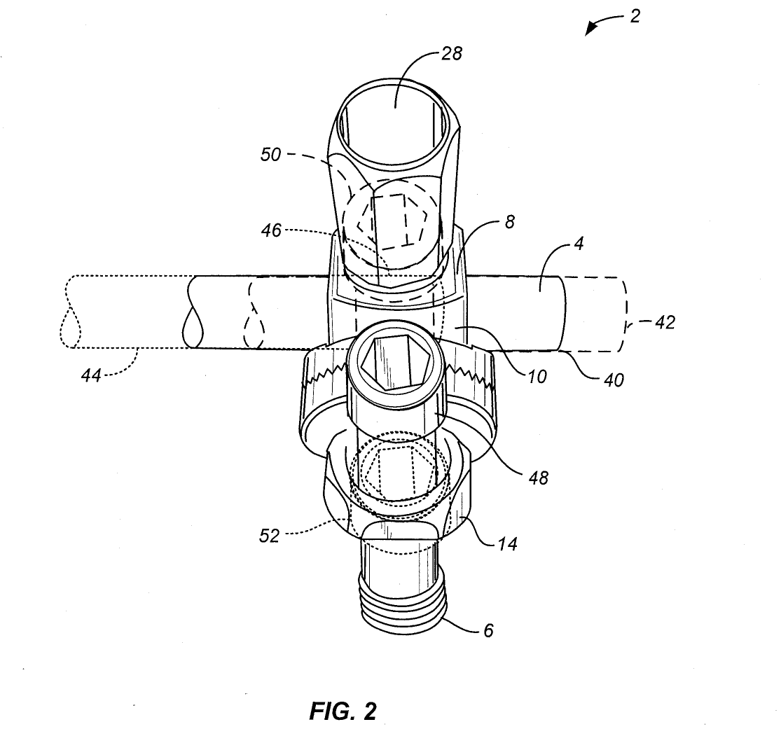

The present cap invention facilitates the pre-operative construction of the spinal system assembly. When the spinal system assembly is pre-assembled with the cap 56 already on the rod 4, the present invention makes it more convenient for the surgeon and reduces his/her time putting the assemblies together during the operation. With such caps, spinal system assemblies will also not separate before or after spinal compression and final tightening. Specifically, the present cap invention prevents the connector assembly 8 from reaching the most critical position 44 (dotted line) shown in FIG. 2. More specifically, a cap 56 will prevent the connector assembly 8 from reaching the end of the spinal rod 4 and coming completely apart from its rod 4. In brief, a cap meets the previously mentioned need to prevent rod and connector disassembly especially during the intra-operative period.

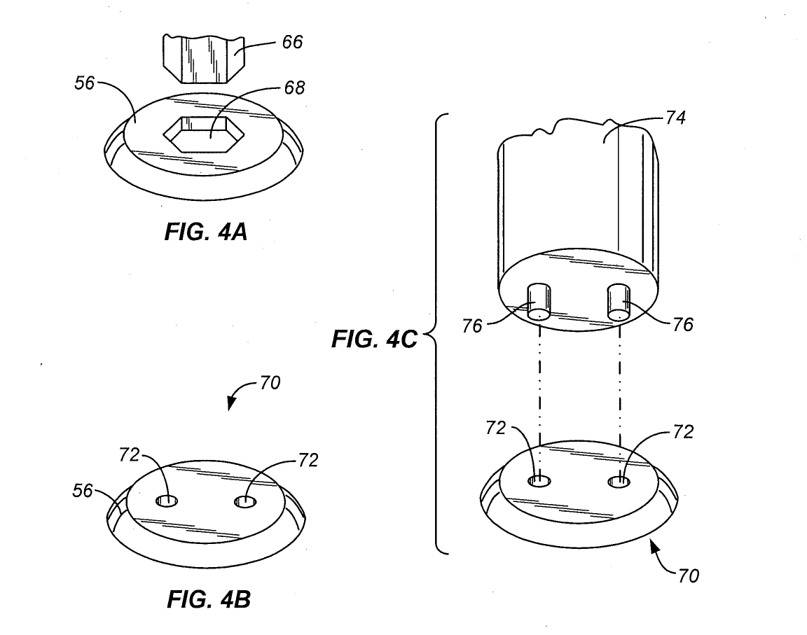

If a permanent cap is desired, a series of cap attachments and driver embodiments are presented in FIG. 4 to tighten the cap to the rod threads. Such cap attachments and driver embodiments maximize the torque strength and attachment between the cap 56 and spinal rod. In one preferred driver embodiment, a hex driver 66 (male) is placed into a hex slot 68 (female) that lies on top of the present cap invention. The hex driver 66 achieves greater torque force through either a handle or drill attached to it. If removal of the cap is desired, a hex driver 66 can also be used to remove the cap 56.

In another driver embodiment, FIG. 4B shows a pin and hole cap 70 where the cap contains at least two pin holes 72 to provide torque leverage. A pin driver 74 with corresponding males pins 76 are inserted into the pin holes (female) 72 and are torqued to tighten the pin hole cap 70.

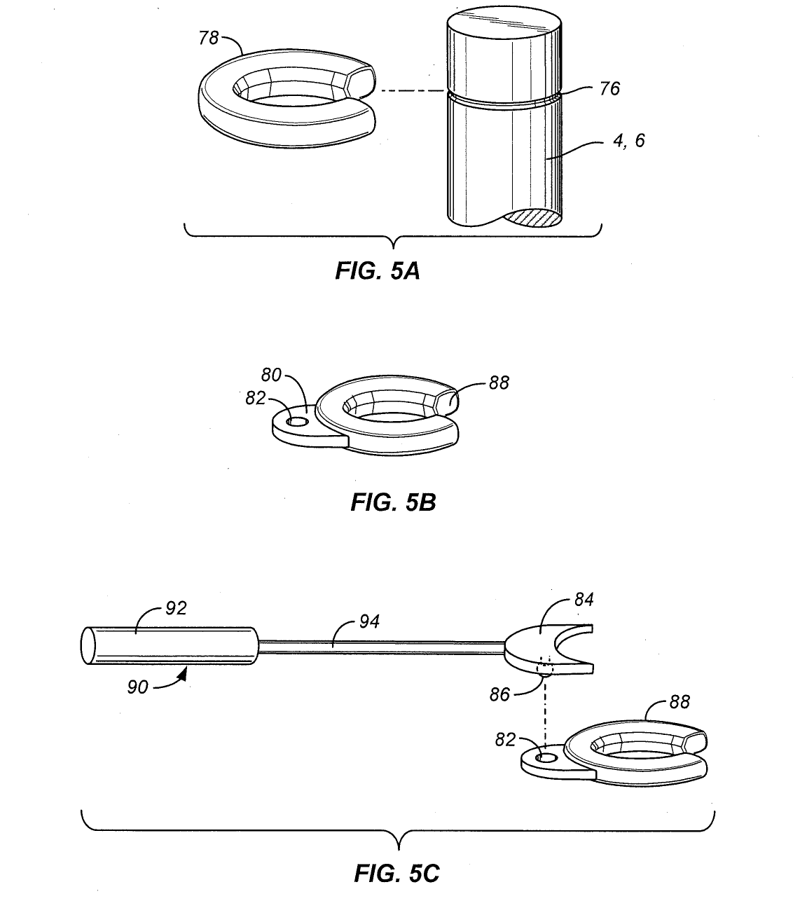

In another cap embodiment, a groove 76 shown in FIG. 5A is cut at or near the end of the spinal rod 4 or bone screw 6. The depth of such groove 76 will depend upon the diameter of the rod so as not to jeopardize the integrity or strength of the rod. In most cases, 1/32 or 1/16 of an inch depth will suffice. In turn, a clip such as c-clip 78 can snap into the groove 76. In other embodiments, the groove 76 may be either a v-shaped or rectangular in shape as to maximize its connection with a corresponding, but opposite angled, c-clip 78. Like the previous cap 56 embodiment, the c-clip 78 prevents slippage of either the rod washer 10 or rod 4 which may separate from one another. A c-clip placed on a rod to prevent the rod from slipping into the critical position 44 (FIG. 2) may prevent a complete implant failure of a particularly dangerous placement or slippage along the rod 4 or the rod washer 10.

Another c-clip embodiment is shown in FIG. 5B. To easily place and, if desired, remove the c-clip, the c-clip 88 may possess an extended and depressed lip 80 to help attach and snap the c-clip 78 to a rod 4. In one embodiment, there can be a lip hole 82 centered on the lip 80. If so, a c-plate 84 shown in FIG. 5C with a stick and ball 86 attached under it can be positioned on top of the lip 80. With a little downward force, the stick and ball 86 snaps into the lip hole 82. With the c-plate 84 preventing any side-to side movement of the c-clip 78 and the stick and ball 86 preventing any up and down movement, the lipped c-clip 88 can be easily pushed and snapped into the groove 76 in the rod 4. This c-plate tool 90 may have a handle 92 and a shaft 94 to provide it with more leverage and torque. A slight and angled twist of the handle will easily snap the stick and ball 86 out of the lip hole 82. The combination of a lipped c-clip 88 and c-plate tool 90 would be very convenient when the placement of a c-clip is temporary and needs to be removed quickly during intra-operative procedures especially when placing connector assemblies onto bone screws.

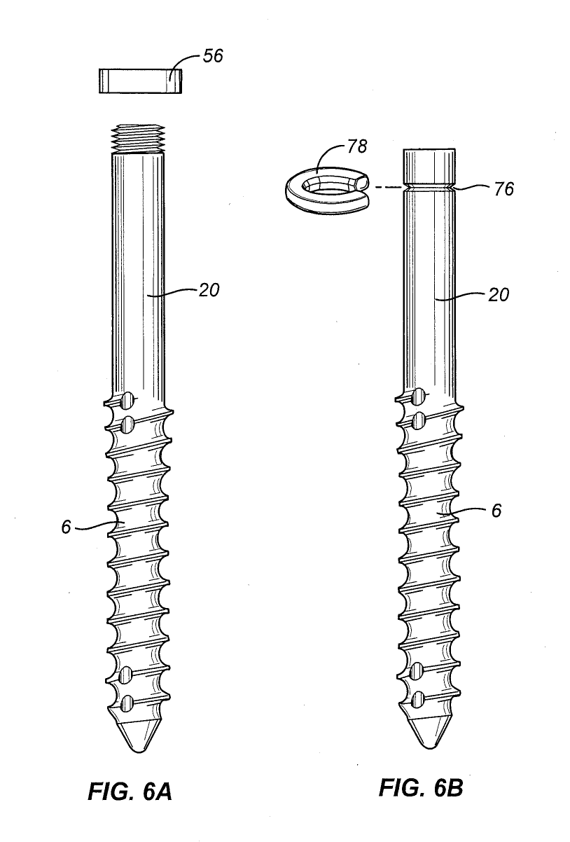

Now referring to FIGS. 6A and 6B, the same cap and c-clip embodiments can be applied to the bone screw 6. FIG. 6A shows a preferred threaded cap 56 embodiment and FIG. 6B shows a preferred c-clip 78 embodiment. These embodiments become particularly important when bone screw 6 possess the variable height shank 20. While the variable height bone screw 6 allows vertical height differences between the rod 4 and the bone screw 6 and their respective rod and bone washers 10, 14 to be met, the cap 56 or the c-clip 78 provides an indication to the surgeon that the maximum height has been reached with particular screw size. If the bone screw washer 14 touches either the threaded cap 56 or c-clip 78 or reaches the critical position 52 illustrated as in FIG. 2, it may be best to consider a longer bone screw 6 to add a measure of slippage clearance and security. If the bone screw 6 is already in place and removal is not desirable, the threaded cap 56 or c-clip 78 gives the surgeon additional confidence that the assembly more than likely will hold even if its position is not optimal. On the other hand, if a bone screw appears to be slipping from its connector assembly 8 and approaching the critical position 52, a surgeon can place either a cap 56 or c-clip 78 through a minimally invasive procedure (e.g., laparoscopic surgery) onto the bone screw 6.

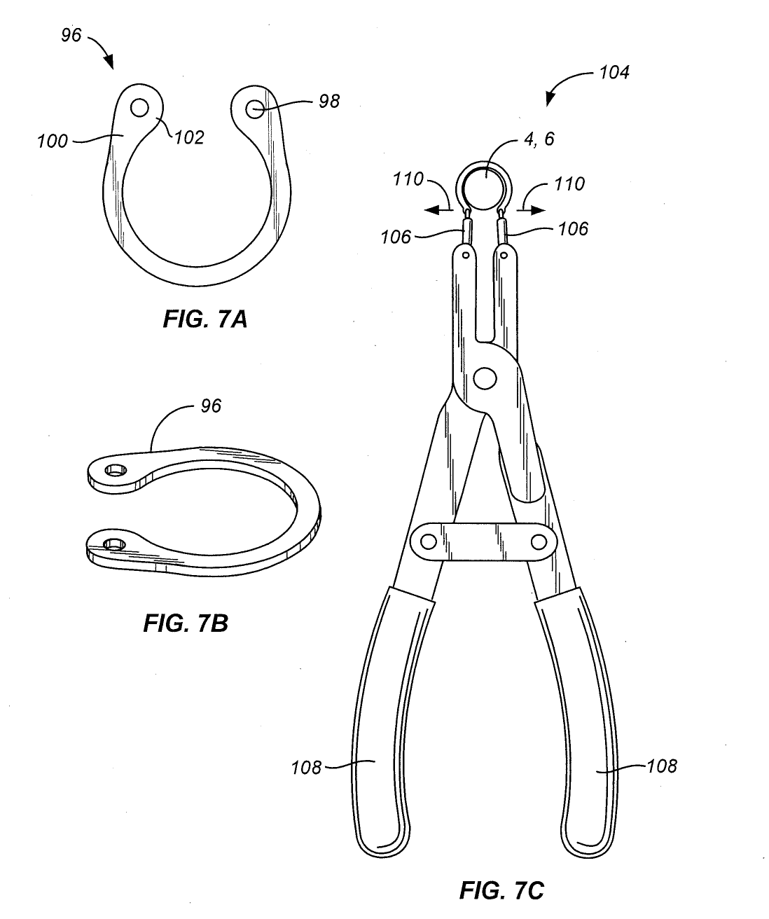

A cap or c-clip placed on the end of a bone screw may hold the bone screw temporarily in place until the connector assembly is fixed by its set screw. Whereas all of the cap, c-clip and lipped c-clip embodiments can be formed for permanent implantation, it might be advantageous to use a light c-clip that can be easily snapped in and out of a groove of a bone screw. Such a light c-clip 96 embodiment is shown in FIG. 7A. The light c-clip in one embodiment contains a pair of arm holes 98 at the end of its arms 100. At the end of the arms 100, the metal may be expanded to form a flange 102. The flange 102 gives the light c-clip 96 and its arm holes 98 a little more strength during its expansion as it is being placed into its bone screw 6 groove shown in FIG. 6B. The ends of the flange 102 may be bent outward to provide easy placement and removal.

A major feature of the light c-clip 96 is its thin metal profile shown as in FIG. 7B. It may be made of stainless steel or titanium like the previous cap 56 and c-clips 78, 88 embodiments. Alternatively, the present light c-clip 96 may be made from light alloys like copper-zinc, aluminum-nickel, copper-aluminum-nickel or nickel-titanium (NiTi). NiTi alloys, for example, can have their transition temperature set below the expected room temperature. This allows the metal to be deformed under stress, yet retain its intended shape once the metal is unloaded again. With alloys, the light c-clip 96 can be spring-like and snapped onto and removed quickly from bone screw 6. It is not intended, however, that the light c-clip 96 be exclusive to just the bone screw 6. If desired, the light c-clip 96 can also be used on the spinal rod 4.

To quickly snap a light c-clip 96 on and off either a bone screw 6 or spinal rod 4, a tool is shown in FIG. 7C. The c-clip expander tool 104 consists of at least two pins 106 that are inserted into the arm holes 98 of the light c-clip 96. As a surgeon squeezes the handles 108, the c-clip expander 104 expands 110 in the opposite direction thereby expanding the light c-clip 96 around a spinal rod 4 or bone screws 6 grooves 76 (FIG. 5A). A release of the handles 108 snaps the light c-clip 96 into the groove 76 of either a spinal rod 4 or bone screw 6.

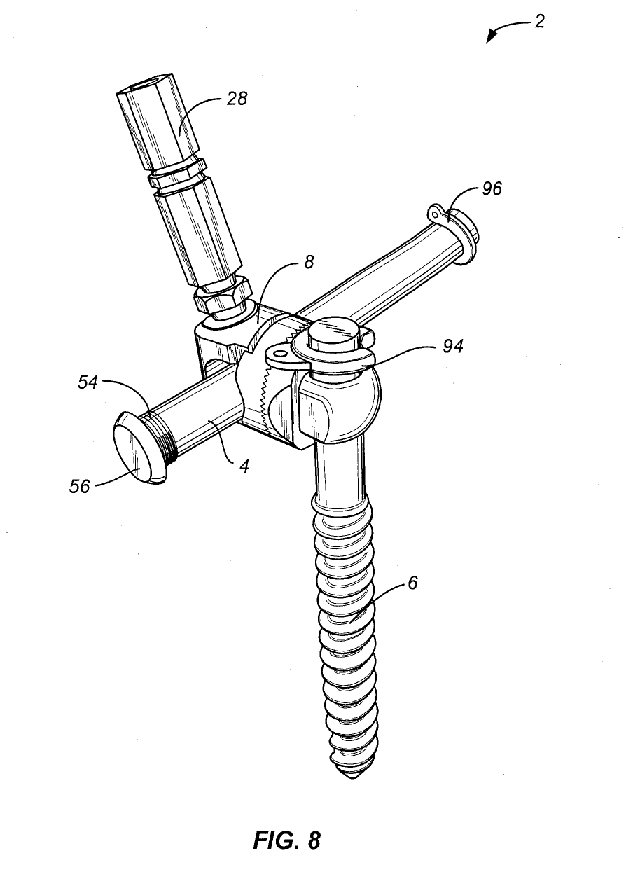

Finally, FIG. 8 shows the cap and c-clip embodiments attached to various positions on a spinal system assembly 2. The cap and c-clip embodiments are not exclusive to their respective position in FIG. 8, but are placed there for illustrative purposes. In one case, the cap 56 is threaded onto the spinal rod threads 54 at the end of a spinal rod 4. A light c-clip 96 may be placed at the other end of the spinal rod 4 if multiple level constructs are anticipated in the future at this end. Such cap or c-clip embodiments will prevent the connector assembly 8 from coming off its spinal rod 4 both intra-operatively or post-operatively. A lipped c-clip 94 is shown on a bone screw 6. In this case, a surgeon may want to temporarily place the lipped c-clip 94 onto the bone screw 6 prior to performing segmental spinal distraction and compression. The lipped c-clip 94 ensures that the connector assembly will not come apart during such a surgical procedure. Once the surgeon obtains the desired spinal curvature, the set screw 28 is tightened to its final torque with the top of the set screw 28 then snapping off. When the spinal system assembly is in place, the surgeon may wish to remove the lipped c-clip 94 or even the light c-clip 96 with the c-plate 90 or c-clip expander 104 tools. With all of the embodiments of the present invention, the need to prevent the connector assembly from coming apart is met.

In the foregoing specification, the invention has been described with reference to specific preferred embodiments and methods. It will, however, be evident to those of skill in the art that various modifications and changes may be made without departing from the broader spirit and scope of the invention. For example, while several caps, c-clips and tool embodiments have been described, those of skill in the art will recognize that alternative means of securing or attaching caps and c-clips could also be used. Accordingly, the specification and drawings are to be regarded in an illustrative, rather than restrictive, sense; the invention being limited only by the appended claims.

What is claimed is:

1. A rod assembly comprising a rod with threads at one end and a cap with interior threads screwed onto the threaded portion of said rod.

2. A bone screw assembly comprising a bone screw with bone screw threads at one end and cap threads at the other end further comprising a cap with interior threaded threads screwed onto the cap threads.

3. The rod assembly of claim 1 wherein said rod assembly cap has a head which is smooth and rounded.

4. The rod assembly of claim 1 wherein said cap has a head with a hex slot in it.

5. The rod assembly of claim 1 wherein said cap has a head with pin holes.

6. A method for placing a cap onto a spinal rod or bone screw comprising the steps of:
selecting a cap with interior threads;
attaching said cap to said spinal rod or bone screw by screwing said cap onto threads formed on said spinal rod or bone screw;
tightening or loosening said cap from said spinal rod or bone screw with or without a driver tool.

7. A rod assembly comprising a rod with a circumferential groove and a c-clip attached to said groove.

8. A bone screw assembly comprising a bone screw with a shank and bone screws threads, wherein a groove is fowled in said shank to receive a c-clip.

9. The rod assembly of claim 7 wherein said c-clip has a lip and hole.

10. The rod assembly of claim 7 wherein said c-clip has two flange arms with holes.

11. A method for placing c-clips onto a spinal rod or bone screw comprising the steps of:
selecting a c-clip;
forming a circumferential groove in said spinal rod or bone screw;
attaching said c-clip to said spinal rod or bone screw groove by snapping said c-clip onto said groove.

12. The method of claim 11 wherein a medical tool is used to snap said c-clip into said groove.

13. The method of claim 12 wherein said medical tool comprises a shaft, c-plate, stick and ball.

14. The method of claim 12 wherein said medical tool has two pins that can be inserted into flange holes on said c-clip.
